(19) United States
(12) Patent Application Publication
(10) Pub. No.: US 2011/0208192 A1
(43) Pub. Date: Aug. 25, 2011

(54) SURGICAL TOOL, ESPECIALLY FOR MACHINING BONES FOR INSERTION OF A DENTAL IMPLANT

(75) Inventors: Ady Palti, Baden-Baden (DE); Herbert Hatzlhoffer, Waldbronn (DE)

(73) Assignee: GPI TECHNOLOGY GMBH, Baden-Baden (DE)

(21) Appl. No.: 12/991,522

(22) PCT Filed: May 9, 2008
(86) PCT No.: PCT/EP2008/003737

§ 371 Date: May 4, 2011

(51) Int. Cl. A61B 17/16

(52) U.S. Cl. 606/79

(57) ABSTRACT

The description, among other things, concerns a Surgical Tool for the preparation of bones, comprising a tool shank, designed to be inserted into a rotatable tool holder and having a duct for conducting the flow of a cooling or rinsing fluid, a machining part driven by the tool shank and rotatable around an axial rotation axis and placed at the proximal axial end of the tool shank, a guide element extending in axial direction of the Surgical Tool, placed at the proximal axial end of the machining part, axially movable relative to the machining part and designed for insertion into a pilot drilling, having at its surface one or several rinsing slots extending in axial direction for conducting the cooling or rinsing fluid, wherein the Surgical Tool and the rinsing slots are designed in such a manner that the exit point at which the cooling or rinsing fluid exits the duct into the rinsing slots, is placed in the area of the passage between the proximal axial end of the machining part and a distal axial area of the guide element.

The invention relates to a surgical tool for preparing bones, especially for machining bones for the insertion of a dental implant.

The tool according to the invention can be used in all areas of bone surgery. Without restricting generality, it is described below on the basis of the example of implant drillings for jaw surgery.

Dental implants are foreign bodies inserted into the jawbone. The subarea of odontology that is concerned with the implanting of dental implants in the jawbone is referred. to as implantology. By virtue of being suitable for use as a carrier of a tooth replacement, dental implants assume the function of artificial tooth roots. For drilling the holes for setting the dental implants in the jaw, a drilling template is used.

In the meantime, the technique of replacing a lost tooth by a dental implant and a dental prosthesis or bridge fastened on it has become established practice. An implant produced from ceramic compound or metal and anchored in the bone, known as the implant root, is used for this, and the artificial tooth crown is fastened on it. For this purpose, a. bore for the implant root must be introduced into the bone at the location of the lost tooth. Since the artificial tooth crown must fit harmoniously into the row of teeth, the implant root must have as large a. diameter as possible for better absorption of masticating forces and the amount of bone available in the jaw is limited, the position and angular orientation of the bore must be exactly precalculated and maintained.

In order to ensure this, a drilling template is usually created first, having at the predetermined location a drilling sleeve which is adjusted in its angular orientation and the inside diameter of which corresponds to the diameter of a pilot drill for the jaw drilling. The drilling template is held by the patient during the drilling of the pilot bore. This drilling template can be produced on the basis of a jaw model of the patient or purely from data obtained by radiology or computer-assisted tomography. Furthermore, the information concerning the extent of the jaw bone that is necessary for establishing the drilling direction is obtained by means of computer-assisted tomography, various sectional representations through the jaw being possible. Other methods which use measurement of the jaw for producing a drilling template are, for example, so-called bone mapping, bone measurement with a probe or other methods of measurement.

Drilling templates are therefore aids for assisting the implantologist to introduce a bore into a patient's jawbone into which the implant is to be inserted. The drilling template has a borehole, which is created on the basis of the jaw model and serves as a guide for the drill during the introduction of the bore or pilot bore into the jawbone. The borehole should have the correct position and angular orientation.

Before an implant is introduced into a bone, the bone material is first machined with special surgical tools. Often a pre-drilling, known as pilot drilling, is first performed with a relatively thin drill, with which the preparation depth is ensured by depth limiting elements. After that, the pre-drilling channel is drilled open with the aid of a so-called shaping drill and is thereby given the shape necessary for the implant. In a next step for preparing the bone, the shaped bore is provided with a thread. For this purpose, a thread cutter is screwed into the bore.

The shaping drill therefore serves for drilling the bore into the jawbone after the pilot drilling has been carried out with a pilot drill (and possibly the guiding sleeves of a drilling template). During the drilling with the shaping drill, generally no guiding sleeve is used.

During this operation, considerable difficulties may be encountered with the conventional machining tools. There is always the risk of deviating from the predetermined drilling channel during the machining, and damaging surrounding bone material. This applies in particular in the preparation of jawbones, because the implant bore extends into the porous spongy bone material of the spongiosa and, under some, circumstances, the tool penetrates into the sinuses of the bone tissue. As a result, the patient's jaw would be greatly damaged and possibly even make the insertion of implants impossible.

A further risk is that the machining goes beyond the prescribed depth of the bore and thereby breaks through the bottom of the bore, damaging both jaw material and nerves.

In the prior art, EP 1 304 087 A2 discloses a surgical tool for preparing bones, especially for machining bones for the insertion of a dental implant, which comprises
a tool shank, which is designed for being clamped into a rotatable tool holder and has a channel for guiding a cooling or flushing fluid through,
a machining part, which is rotatable about an axial axis of rotation, can be driven by the tool shank and is arranged at the proximal axial end of the tool shank, and
a guiding element, which extends in the axial direction of the tool, is arranged at the proximal axial end of the machining part, can be moved axially in relation to the machining part and is designed for being introduced into a pilot bore, and has on its surface one or more axially extending flushing grooves for guiding the cooling or flushing fluid.

After the pilot drilling, such a shaping drill is used to perform the final drilling (known as “finish drilling”) before the insertion of the implant body into the jaw. The shaping drill, which can also be referred to as a telescopic drill, has the property of being guided by the guiding element, which is attached to the active cutting edge, is telescopically extendable and retractable in the axial direction and, when pressed, disappears inside the drill head. During use, the drill is pressed into the pilot bore, without being tilted as this happens. When the guiding element reaches the bottom of the pilot bore, it presses against a spring force into the drill head, until it reaches a stop. When the guiding element in this stop position protrudes from the drill head, for example by 0.5 mm, and is not cutting, the drill head remains at a standstill in its drilling movement in the blind hole of the pilot bore and can no longer be actively guided any further. The “finish drilling” has consequently been carried out.

Even though it has been possible to achieve good results with this known tool, it has been found in practice that both the cooling of the machining part by the cooling fluid and the removal of the bone chips, tissue fragments and blood from the drilling location during the drilling are not optimal and should be improved.

Against this background, the present invention is based on the object of providing a surgical tool with which bone material can be machined without'the tool suddenly deviating from the drill channel or penetrating the drill channel, improved cooling and improved removal of bone chips, tissue fragments and blood being made possible.

The object is achieved according to the invention by a surgical tool with the features of patent claim 1. Preferred refinements are provided by the dependent patent claims and the following description with associated drawings.

A surgical tool according to the invention for preparing bones, especially for machining bones for the insertion of a dental implant, therefore comprises
a tool shank, which is designed for being clamped into a rotatable tool holder and has a channel for guiding a cooling or flushing fluid through,
a machining part, which is rotatable about an axial axis of rotation, can be driven by the tool shank and is arranged at the proximal axial end of the tool shank, and
a guiding element, which extends in the axial direction of the tool, is arranged at the proximal axial end of the machining part, can be moved axially in relation to the machining part and is designed for being introduced into a pilot bore, and has on its surface one or more axially extending flushing grooves for guiding the cooling or flushing fluid,
   and has the special feature that the tool and the flushing grooves are formed in such a way that the outlet point at which the cooling or flushing fluid leaves the channel and enters the flushing grooves is arranged in the region of the transition between. the proximal axial end of the machining part and a distal axial region of the guiding element.

It has been found that optimum cooling and flushing is possible with such a surgical tool. The reason for this is that the fluid used for cooling or flushing in the case of a tool according to the invention leaves directly at the proximal cutting edge of the machining part, and not, as in the prior art, at the tip of the guiding element at the bottom of the pilot bore. This has the advantage of better cooling of the cutting edge, and consequently a machining or drilling operation that does not harm the jaw or tissue, because the cooling fluid does not leave only once it reaches an opening located at the proximal end of the guiding element, having to flow through the narrow gap between the proximal end of the guiding element and the bottom of the pilot bore lying at the latter and back through the flushing grooves to the proximal end of the machining part, but can leave directly at the proximal end of the machining part. Better flushing of bone chips, tissue fragments and blood is thereby obtained by the flow being directed radially from inside to outside, and the bone chips that are carried away from the drilling location consequently being transported away radially outward. As a result, the outlets of the cooling channels and the cooling channels or flushing channels themselves become clogged to a lesser extent.

A further advantageous feature in this connection may be that the surgical tool and the flushing grooves are formed in such a way that the cooling or flushing fluid flows in the flushing grooves in the direction of the proximal end of the guiding element. The guiding element therefore has one or more axially running outer grooves, which may also be referred to as slots or channels, in which the cooling fluid flows in the direction of the proximal tip of the guiding element. In the prior art, on the other hand, the cooling fluid leaves at the proximal tip of the guiding element and runs back into the flushing grooves on the outside of the guiding element. In the case of the invention, therefore, the flow of the cooling or flushing fluid in the flushing grooves of the guiding element is proximally directed, whereas in the prior art it is distally directed.

It may accordingly be provided in an advantageous refinement that the guiding element does not have a channel for guiding the cooling or flushing fluid through and/or that the guiding element does not have at its proximal end an opening from which the cooling or flushing fluid leaves.

According to a further advantageous feature, it is proposed that the machining part and the tool shank together form a one-piece part. In the case of EP 1 304 087 A2, the drill head and the tool shank are two separate parts and the drill head is screwed onto the tool shank as far as a stop by means of a threaded connection. The screwed connection according to the prior art represents a weakness, that is from a hygienic viewpoint, since bone chips, tissue fragments and blood can accumulate in the fine thread, and also from a mechanical viewpoint, because this may represent a possible rupture point or the connection may come undone, in particular if a number of drillings are being carried out with the drill on a patient, for example eight drill holes, the drill undergoing considerable loading in the hard bone. The one-piece configuration of the machining body (for example drill head or threaded cutting shank) with the tool shank (for example drill shank), the two parts being produced together from one piece, avoids these disadvantages. At the same time, the rupture resistance of the construction is ensured even with very small dimensions.

One or more or all of the parts comprising the tool shank, the machining part and the guiding element may, for example, consist of industrial steel numbers 1.4301, 1.4303 or 1.4305. Industrial steels with the numbers 1.4034 and 1.4197 are also advantageous. The steel 1.4197 has the advantage that it is made somewhat more “pliable” than the steel 1.4034 by tempering, but at the same time is also less hard/holds its edge less well. In the case of thin drills, for example a pilot drill for pre-drilling, therefore, the steel 1.4197 could rather be used instead of the steel 1.4034. A particularly advantageous embodiment, especially when the machining part and the tool shank are formed together in one piece, may be that one or more or all of the parts comprising the tool shank, the machining part and the guiding element consist of titanium, titanium nitrite, titanium nitride, zirconium, zirconium oxide or ceramic. These materials have the advantage that they are biologically neutral, by contrast with steel, to which many patients are allergic. Consequently, even such allergic patients can be treated.

An additional advantageous form may consist in that the sum of the cross-sectional areas of the flushing grooves in the guiding element is at least as great as the cross section of the channel in the tool shank. The advantage of this is that it prevents a reduction of the flow cross section for the flowing of the cooling or flushing fluid from being caused by a cross-sectionally constricted transition from the channel into the flushing grooves, and consequently prevents a reduction of the coolant flow.

The invention is explained in more detail below on the basis of exemplary embodiments that are represented in the figures. The special features described therein can be used individually or in combination with one another to create preferred refinements of the invention. In the figures:

FIG. 1 shows a side view of a drill according to the invention with a cylindrical drill head,

FIG. 2 snows a basic axial section of the drill shank and the machining part of the drill from FIG. 1,

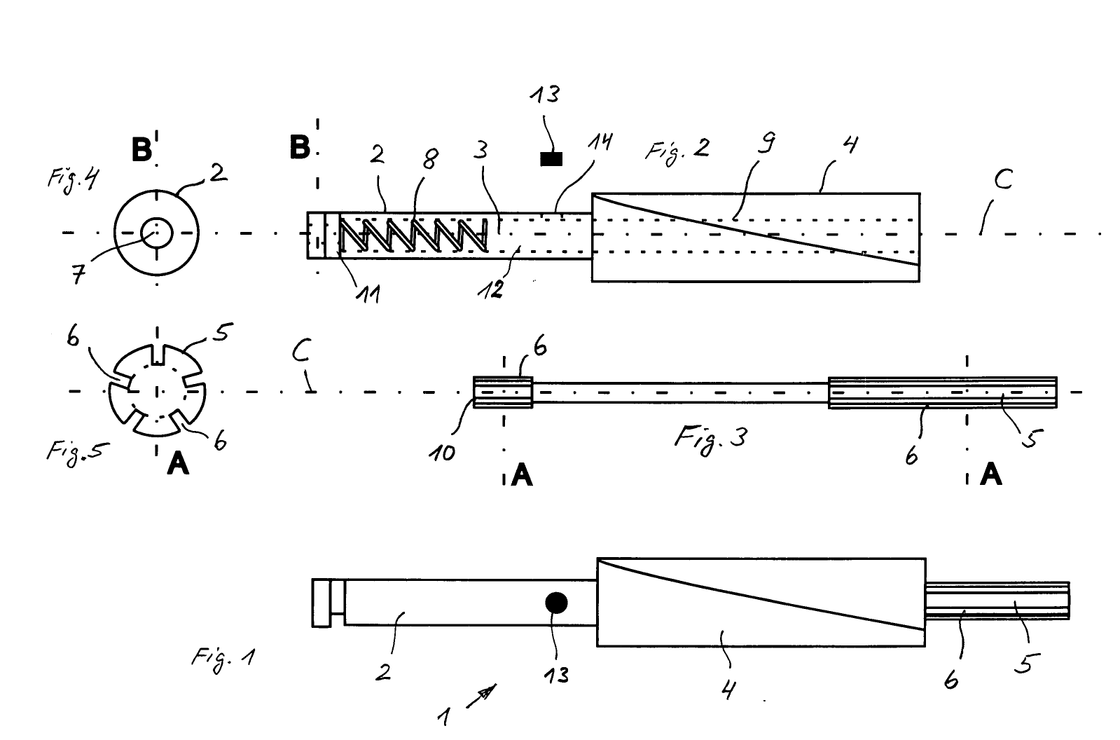

FIG. 3 shows a side view of the guiding element of the drill from FIG. 1,

FIG. 4 shows a section B taken from FIG. 2,

FIG. 5 shows a section A taken from FIG. 3,

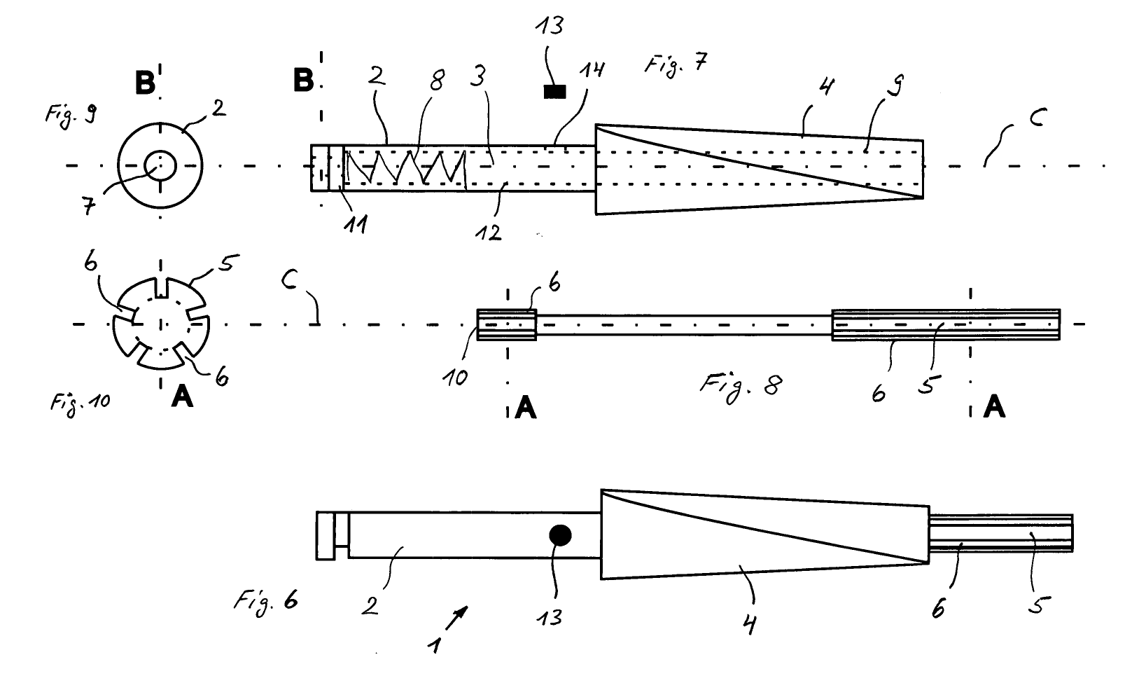

FIG. 6 shows a side view of a drill according to the invention with a conical drill head,

FIG. 7 shows a basic axial section of the drill shank and the machining part of the drill from FIG. 6,

FIG. 8 shows a side view of the guiding element of the drill from FIG. 6,

FIG. 9 shows a section B taken from FIG. 7 and

FIG. 10 shows a section A taken from FIG. 8.

In FIGS. 1 to 5, a first exemplary embodiment of a tool 1 according to the invention is represented in the form of a drilling device for drilling a hole into a jaw for the insertion of a dental implant. The drilling device comprises a tool shank 2, which is designed for being clamped into a rotatable tool holder and has a channel 3 for guiding a cooling or flushing fluid through, as well as a machining part 4, which is rotatable about an axial axis of rotation. C, can be driven by the tool shank 2 and is arranged at the proximal axial end of the tool shank 2. In this exemplary embodiment, the machining part 2 is cylindrically formed and is, for example, a drill, especially a twist drill, or a countersinking drill.

The machining part 4 preferably has an outside diameter of between 1.8 mm and 15 mm, preferably between 2.0 mm and 12 mm, particularly preferably between 2.5 mm and 10 mm. The lower limit is based on the requirement that, because of the internal parts described below, the remaining wall thickness must be great enough for the cutting edge. The upper limit is based on expedient ranges of general orthopedic applications.

Furthermore, the tool 1 comprises a guiding element which extends in the axial direction of the tool, is arranged at the proximal axial end of the machining part 4, can be moved axially in relation to the machining part 4 and is designed for being introduced into a pilot bore, and has on its surface one or more axially extending flushing grooves 6 for guiding the cooling or flushing fluid. The outside diameter of the guiding element 5 is as great as or slightly greater than the inside diameter of the pilot bore. Expedient values lie between 0.5 mm and 6.0 mm, preferably between 0.8 mm and 4.0 mm, particularly preferably between 1.0 mm and 2.5 mm.

The tool 1 and the flushing grooves 6 are formed in such a way that the outlet point at which the cooling or flushing fluid leaves the channel 4 and enters the flushing grooves 6 is arranged in the region of the transition between the proximal axial end of the machining part 4 and a distal axial region of the guiding element 5.

After being clamped into a drive that is not represented, for example an angle piece, the tool shank 2, for example a tool head carrier, is set in rotation and transfers the rotational movement to the machining part 4, which rotates about the axis C as a rotating machining part. The machining part may be fastened on a carrier, for example a tool head carrier.

At the beginning of the machining, the guiding element 5 or its tip is inserted into the pre-drilling channel drilled by the pilot drill or, in the case of thread-cutting, into what still remains of the pre-drilling channel. The rotating machining parts, for example shaping drills or thread cutters, then work themselves into the bone along the guiding element 5 during the machining of the bone. As this happens, they are displaced axially along the guiding element 5 in a defined manner and at no time is there any risk of them suddenly deviating to the side.

It may advantageously be provided that the guiding element 5, or at least the proximal part of the guiding element 5 that can be introduced into a pilot bore, is formed in the manner of a pin. Furthermore, it advantageous if the guiding element 5, or at least the proximal part of the guiding element 5 that can be introduced into a pilot bore, is not designed for the material-removing machining of bones, in particular is not designed for drilling, cutting or milling. A non-cutting guiding element 5 ensures that the user can drill an exactly round hole, without deviating from the pilot bore with regard to depth and/or angle.

A particularly advantageous refinement may consist in that the maximum machining depth can also be predetermined. by the guiding element 5. This ensures that the machining parts cannot penetrate into the bone beyond the predetermined bore. The machining space for the rotatable machining part 4 is exactly predetermined and there is neither the risk of lateral deviation nor the risk of exceeding the depth of the bore. Therefore, sources of error during machining are eliminated to the greatest extent.

If the guiding element 5 predetermines the maximum working depth, this is accompanied by many advantages. For instance, depth stop rings do not have to be laboriously provided above the drill. Furthermore, the machining is automatically stopped at the desired machining depth and it is not necessary to measure off the depth of penetration on a linear scale before or during the entire machining operation. The guiding element 5 allows a maximum machining depth to be predetermined even when the machining part 4 has still not yet penetrated completely into the bore. This is not possible when using conventional depth stop rings, since these must generally be fastened above the machining part 4 and, as a result, would not butt against the mouth of the bore at all.

The maximum machining depth may either be predetermined by the guiding element 5 according to the production specification or, if need be, be set on the tool itself by the user according to his wishes. The maximum machining depth can be chosen as desired, so that for example only the upper part of the bore in the bone is machined or the machining takes place almost up to the end of the bore.

If the maximum machining depth is predetermined by the guiding element 5, it is conceivable, for example, that the machining depth can be predetermined by a stop. In this case, the rotatable machining part 4 can only be displaced axially on the guiding element 5 as far as this stop. Machining beyond the stop is then not possible.

In a first embodiment, the machining part 4 can rotate about the guiding element 5, the guiding element 5 remaining in its position without undergoing rotary movements of its own and only the machining part performing rotational movements.

In a further embodiment of the tool 1 according to the invention, the machining part 4 rotates with the guiding element 5, it being possible for the rotational movement to be transferred from the guiding element 5 to the machining part 4, or vice versa.

The transfer of the rotational movement may take place for example by a positive connection in the direction of rotation. An axial movement along the guiding element 5 may in this case be enforced by a mutually wedge-shaped or conical sloping of the positive connection. On account of this sloping, the machining part 4 is displaced in the direction of the bore during the working operation, since the rotary movement is partly transformed by the sloping into a movement with an axial component.

A predetermination of the maximum machining depth is in this case also possible by the positive connection between the guiding element 5 and the machining part 4 being releasable as from a certain depth and, at this depth, the machining part 4 no longer rotating and as a result remaining for a time in the bone. In this case, ineffectual turning of the guiding element 5 occurs, no rotational movement being transferred any longer from the guiding element 5 to the machining part 4.

It is possible that, after the machining part 4 has remained for a time in the bone and the rotational movement of the guiding element 5 has been stopped, a new positive connection with the guiding element 5 is established and, as a result, the machining part 4 is removed from the bone. For example, this may involve the machining part 4, for example a thread-cutter, being unscrewed from the bore again in a rotary movement in the opposite direction.

Various machining parts 4 can be used for the surgical tool 1 according to the invention. On the one hand, drills, especially shaping drills, and, on the other hand, thread-cutters have proven to be particularly important.

During the machining operations, bone material is removed. Apart from the bone chips, tissue fragments and blood are also present. These contaminants should be removed from the bore already during the working operation, since smearing can otherwise occur. For this purpose, cooling or flushing fluid is supplied through the inlet opening 7 in the extreme distal end of the tool shank 2. The cooling or flushing fluid runs through the shank of the drill into the drill head and is discharged from the drill tip by way of the flushing grooves 6 provided on the guiding element 5. This ensures that the fluid leaves at the point at which the proximal cutting edge of the machining tool 4 is at its most active.

The cooling or flushing fluid introduced during the machining operation is, for example, an isotonic saline solution. The aqueous solution serves on the one hand for cooling the machining tool; on the other hand, the removed parts of the bone are thereby swept out of the bore in the bone. In order to make this possible, in the case of the device according to the invention the guiding element 5 is provided with flushing grooves 6. However, the saline solution does not leave at the tip of the guiding element 5 and run out of the bore in the bone again along the flushing grooves 6, as in the prior art, but instead the fluid already leaves in the region of the transition between the proximal axial. end of the machining part 4 and a distal axial region of the guiding element 5, i.e. at the distal end and riot at the proximal end of the pilot bore. The bone material is flushed out along with the fluid, so that smearing of the bore drilled by the tool 1 cannot occur.

The flushing grooves 6 on the guiding element 5 may extend up to the proximal end of the guiding element 5, in order also to cool or flush the guiding element 5 and the pilot bore. It is in fact difficult for the saline solution to flow into and out again from the pilot bore in the bone, since the guiding element 5 generally fits in the pilot bore with little play. However, it has been found that cooling or flushing of the pilot bore achieved in this way is sufficient, because it is more important to cool and flush the proximal end of the machining part 4 than the guiding element 5 in the pilot bore. Nevertheless, in the case of the invention, an aqueous solution passing through does not cause a build-up of pressure, which could in the worst case lead to the tool 1 being forced out of the bore or pilot bore or could have the effect that the cooling channel in the tool 1 is no longer flowed through and, as a result, undesired heating up occurs. This is undesired because overheating of the tool could cause considerable harm to the patient, since bone material and nerves embedded therein would be severely damaged.

An axially acting spring element 8, which presses the guiding element 5 out of the machining part 4, is expediently provided between the machining part 4 and the guiding element 5. The tool 1 therefore has in this case a spring element 8, which produces a restoring force that moves the guiding element 5 in the proximal direction with respect to the machining tool 4, the guiding element 5 being movable axially against the restoring force. The advantage of using the spring force for pressing out the guiding element 5 is that the tool 1 is immediately ready for use. The guiding element 5 does not first have to be withdrawn from the tool 1 to then be introduced into the pilot bore. The spring force has the effect that the guiding element 5 is automatically brought out as far as a stop, and can then be inserted directly into the pilot bore. Furthermore, there is no danger of the guiding element slipping back again into the tool 1, for example during the machining operation. Consequently, the user can even work against gravitational force without having to fear that the guiding element 5 will disengage from the pilot bore and thereby be adversely affected in its guiding function.

The distal end piece of the tool shank 2, which is clamped into a tool holder, is for example a DIN-standard part or finished part. The machining part 4 is fixed on the tool shank 2 by means of a thread or, according to a preferred embodiment, is formed in one piece with the tool shank 2. The machining part 4 may be screwed onto the tool shank 2 as far as a stop. The end of the tool shank 2 thereby also butts against a stop in the interior of the machining tool 4. The guiding element 5 is mounted displaceably in the inner bore 9 of the machining part 4. Before the tool is used, the stop of the guiding element 5 may butt against a stop of the machining part 4. The guiding element 5 is in this case pressed against the stop by the spring element 8.

The spring 8 rests at its one end on the spring seat 10 of the guiding element 5. At its other end, the spring rests on the spring seat 11 of the tool shank 2, which at the same time serves as a tool carrier. In the tool shank 2, the spring 8 is guided in the inner bore 12 of the tool shank 3.

Once a pilot drilling that has a diameter only insignificantly greater than the guiding element 3 has been performed with a drill, the guiding element 3 is inserted into this pilot bore. The machining part 4, which is fixedly connected to the tool shank 2, preferably in one piece with it, is set in rotation. The guiding element 3 in this case does not rotate along with it, but remains static for a time in the pilot bore. By exerting a force, the rotating machining part 4 is pressed proximally in the direction of the pilot bore. The opposing force applied by the spring 8 must be overcome thereby. The spring 8 is compressed during this operation. During the drilling operation, the machining part 4 is displaced along the guiding element 3, or the guiding element 3 is displaced with respect to the machining part 4, until a stop is reached. This stop has the effect of predetermining the maximum machining depth to which the rotating machining tool 4 can penetrate into the pilot bore. The spring 8 is compressed during this operation.

A cooling water channel 3 runs through the interior of the tool shank 2 and the machining part 4. An isotonic saline solution is passed through this channel 4 for cooling the tool 1 and for flushing out the removed bone parts. During the processing operation, the isotonic saline solution leaves at the proximal end of the machining part 4. Tip of the guiding element 3 and then runs in the radial direction laterally outward from the outlet point and along the flushing grooves 6 in the proximal direction on the guiding element 3.

Especially in the one-piece form of the tool shank 2 and the machining part 4, a securing element is provided for fastening the guiding element 3 and is used to hold the guiding element 3 in the machining part 4 after it has been inserted into the machining part 4. Serving for this purpose in the exemplary embodiment is a securing bolt 13, which is inserted or screwed into a corresponding radial opening 14 and engages in a corresponding indentation or stepped recess in the guiding part 3. In other embodiments, the guiding element 5 may also have, for example, fastening parts formed as pivotable barbs, which swing out when the guiding element 5 is fitted into the machining part 4 and provide the fixing of the guiding element 5 in the machining part 4.

In FIGS. 4 and 5 it can be seen that, in the exemplary embodiment, an inlet opening 7 or a bore for passing cooling or flushing fluid through the surgical tool 1 has been introduced into the middle of the tool carrier 2 and a total of four flushing grooves 6 have been milled into the guiding element 4.

In FIGS. 6 to 10, a second exemplary embodiment of a tool 1 according to the invention is represented in the form of a drilling device for drilling a hole into a jaw for the insertion of a dental implant. It corresponds to the first exemplary embodiment of FIGS. 1 to 5, with the difference that the machining part 4 is not cylindrically formed but conically formed. The machining part 4 may be, for example, a drill head.

To sum up, the surgical tool 1 according to the invention is distinguished by the fact that, as soon as the pilot bore has been set, the user can carry out the working steps which then follow without any major sources of error. With the tool according to the invention there is neither the risk of sudden lateral deviation from the bore nor breaking out downward from the bore. This is ensured by the tool having a guiding element 5 and by the rotating machining part 4 being displaced axially with respect to the guiding element 5 during the machining operation, improved cooling and improved removal of bone chips, tissue fragments and blood being made possible. With the tool 1 according to the invention, it is possible even for novices to carry out the necessary machining steps for the preparation of bones, especially for the insertion of dental implants.

### LIST OF DESIGNATIONS

1 surgical tool
2 tool shank
3 channel
4 machining part
5 guiding element
6 flushing groove
7 inlet opening
8 spring element
9 inner bore in 4
10 spring seat on 5
11 spring seat on 2
12 inner bore in 2
13 securing bolt
14 opening
C axis of rotation

1. A surgical tool for preparing bones, comprising
a tool shank, which is designed for being clamped into a rotatable tool holder and has a channel (3) for guiding a cooling or flushing fluid through,
a machining part, which is rotatable about an axial axis of rotation, can be driven by the tool shank and is arranged at the proximal axial end of the tool shank,
a guiding element, which extends in the axial direction of the surgical tool, is arranged at the proximal axial end of the machining part, can be moved axially in relation to the machining part and is designed for being introduced into a pilot bore, and has on its surface one or more axially extending flushing grooves for guiding the cooling or flushing fluid,
wherein the surgical tool and the flushing grooves are formed in such a way that the outlet point at which the cooling or flushing fluid leaves the channel and enters the flushing grooves is arranged in the region of the transition between the proximal axial end of the machining part and a distal axial region of the guiding element.

2. The surgical tool as claimed in claim 1, wherein the surgical tool and the flushing grooves are formed in such a way that the cooling or flushing fluid flows in the flushing grooves in the direction of the proximal end of the guiding element.

3. The surgical tool as claimed in claim 1, wherein the guiding element does not have a channel for guiding the cooling or flushing fluid through.

4. The surgical tool as claimed in claim 1, wherein the guiding element does not have at its proximal end an opening from which the cooling or flushing fluid leaves.

5. The surgical tool as claimed in claim 1, wherein the flushing grooves extend up to the proximal end of the guiding element.

6. The surgical tool as claimed in claim 1, wherein the sum of the cross-sectional areas of the flushing grooves in the guiding element is at least as great as the cross section of the channel in the tool shank.

7. The surgical tool as claimed in claim 1, wherein the guiding element, or at least the proximal part of the guiding element that can be introduced into a pilot bore, is formed in the manner of a pin.

8. The surgical tool as claimed in claim 1, wherein the guiding element, or at least the proximal part of the guiding element that can be introduced into a pilot bore, is not designed for the material-removing machining of bones, in particular is not designed for drilling, cutting or milling.

9. The surgical tool as claimed in claim 1, wherein it has a spring element, which produces a restoring force that moves the guiding element in the proximal direction, the guiding element being movable axially against the restoring force.

10. The surgical tool as claimed in claim 1, wherein the machining part and the tool shank together form a one-piece part.

11. The surgical tool as claimed in claim 1, wherein it comprises a securing element, with which the guiding element is held in the machining part after it has been inserted into the machining part.

12. The surgical tool as claimed in claim 1, wherein one or more or all of the parts comprising the tool shank, the machining part and the guiding element consist of industrial steel numbers 1.4301, 1.4303, 1.4305, 1.4034 or 1.4197.

13. The surgical tool as claimed in claim 1, wherein one or more or all of the parts comprising the tool shank, the machining part and the guiding element consist of titanium, titanium nitrite, titanium nitride, zirconium, zirconium oxide or ceramic.

14. The surgical tool as claimed in claim 1, wherein it is formed in such a way that a maximum machining depth can be predetermined by means of the guiding element.

15. The surgical tool as claimed in claim 1, wherein it has a stop for predetermining the machining depth.

16. The surgical tool as claimed in claim 1, wherein the machining part is rotatable about the guiding element.

17. The surgical tool as claimed in claim 1, wherein the machining part is rotatable with the guiding element.

18. The surgical tool as claimed in claim 1, wherein the machining part is formed as a drill, especially as a twist drill, or as a countersinking drill.

19. The surgical tool as claimed in claim 1, wherein the machining part is formed as a thread-cutter.

20. The surgical tool as claimed in claim 1, wherein the machining part is cylindrically formed.

21. The surgical tool as claimed in claim 1, wherein the machining part is conically formed.

22. The surgical tool as claimed in claim 1, wherein the machining part has an outside diameter of between 1.8 mm and 15 mm, preferably between 2.0 mm and 12 mm, particularly preferably between 2.5 mm and 10 mm.

23. The surgical tool as claimed in claim 1, wherein the guiding element has an outside diameter of between 0.5 mm and 6.0 mm, preferably between 0.8 mm and 4.0 mm, particularly preferably between 1.0 mm and 2.5 mm.
